(19) United States
(12) Patent Application Publication
(10) Pub. No.: US 2022/0022515 A1
(43) Pub. Date: Jan. 27, 2022

(54) Composition, Food Supplement and Method for Supporting and/or Improving Intestinal Health

(71) Applicant: NUTRIBAM BV, Malle (BE)

(72) Inventor: Nikolaas VAN RIET, Halen (BE)

(21) Appl. No.: 17/427,900

(22) PCT Filed: Feb. 25, 2020
(86) PCT No.: PCT/IB2020/051589

§ 371 Date: Aug. 2, 2021

(30) Foreign Application Priority Data
Feb. 25, 2019 (BE) 2019/5116
Mar. 5, 2019 (BE) 2019/5134

(51) Int. Cl. A23L 33/125; A23L 33/21; A23L 29/30; A61K 31/702

(52) U.S. Cl. CPC A23L 33/125; A61K 31/702; A23L 29/30; A23L 33/21

(57) ABSTRACT

The present invention relates to a composition for use in supporting and/or improving intestinal health in athletes, comprising an effective dose of at least one human milk oligosaccharide (HMO) from the group of 2′-fucosyllactose (2′-FL), 3-fucosyl lactose (3-FL), lactodifucotetraose (DF-L), lacto-N-tetraose (LNT), lacto-N-neotetraose (LNnT), lacto-N-fucopentaose I (LNFP I), lacto-N-difucohexaose II (LNDFH II), lacto-N-neodifucohexaose II (LNnDFH II), para lacto-N-neohexaose (para-LNnH), lacto-N-neooctaose (LNnO), lacto-N-fucopentaose V (LNFP V), lacto-N-neofucopentaose V (LNnFP V), 3′-sialyl-3-fusoyl lactose (F-SL), LS-tetrasaccharide a (LSTa), 3′-sialyllactose (3′-SL), or combinations thereof.

### TECHNICAL FIELD

The invention relates to intestinal health. In particular, the invention is in the technical area of compositions for supporting and/or improving intestinal health.

### PRIOR ART

Regular exercise and/or sport offers many benefits, including protection against the development of chronic diseases and improved quality of life. These benefits are attributed, among other things, to the anti-inflammatory effects and the strengthening of the neuromuscular function associated with exercise. In addition, intensive sports practice leads to changes in the gastrointestinal microbiota, which is closely related to the sportsperson's well-being. For example, the gastrointestinal microbiota play an important role in the production, storage and consumption of energy obtained from the diet, as well as in inflammation, redox reactions and hydration status.

However, the correlation between exercise and the gastrointestinal microbiota can also be detrimental to persons engaged in sports, especially elite athletes. For example, long and intensive training sessions may lead to the disruption of the gastrointestinal microbiota. As a result, the sportsperson delivers significantly less sports performance and his recovery capacity is drastically reduced. A disrupted gastrointestinal microbiota also reduces the functioning of the immune system, which puts athletes at greater risk of illness, infections, in particular intestinal disorders. In addition, athletes who regularly consume food supplements, especially supplements rich in fructose and/or artificial sweeteners, are at an increased risk of dysregulated gastrointestinal microbiota and infection as these components reach the intestinal tract intact and function as potential nutrients for pathogenic bacteria.

Accordingly, there is a need to support and/or improve intestinal health in athletes in order to support the immune system, minimise the risk of infection, and optimise athletes' sports performance and recovery capacity.

WO 2017 180 501 describes a probiotic composition for improving sports performance and recovery. However, the prebiotic composition of WO '501 has only limited influence on the athlete's immune system and the risk of infections related to the occurrence of pathogenic bacteria in the gastrointestinal tract is little or not affected.

WO 2018 187 792 discloses a 2′-fucosyllactose component and a method of using said component for the treatment of inflammatory intestinal disorders, such as Crohn's disease and ulcerative colitis, or to reduce the risk of relapse in inflammatory intestinal disorders. WO '792, however, emphasises inflammatory intestinal disorders. No indication is given as to how 2′-fucosyllactose can be used non-therapeutically to improve and/or support intestinal health in athletes undertaking long-term and intensive sports performance.

Furthermore, WO 2014/100696 and WO 2018/020473 describe human milk oligosaccharides (HMOs) in relation to intestinal health and cognitive functions. WO 2018/187792 focuses on the use of HMOs in Inflammatory Bowel Disease (IBD). WO 2017/064711 and WO 2017/144062 describe the relationship between HMOs and the intestinal flora, specifically with regard to certain microorganisms, as well as the treatment of intestinal disorders and other disorders. WO 2013/154725 and WO 2017/190754 also describe the use of HMOs in persons with intestinal disorders. WO 2018/024870 and WO 2017/103019 focus on the benefits of HMOs in young children. EP 2 896 628 and EP 2 465 507 discuss the purification of HMOs and describe some applications. None of these documents, however, establish a clear link between HMOs, the athlete and their sports performance.

The object of the present invention is to at least solve some of the above-mentioned problems or disadvantages.

### SUMMARY OF THE INVENTION

To this end, the invention provides a composition for use in supporting and/or improving intestinal health in athletes according to claim 1. The composition comprises an effective dose of at least one human milk oligosaccharide (HMO) selected from the group of 2′-fucosyllactose (2′-FL), 3-fucosyllactose (3-FL), lactodifucotetraose (DF-L), lacto-N-tetraose (LNT), lacto-N-neotetraose (LNnT), lacto-N-fucopentaose I (LNFP I), lacto-N-difucohexaose II (LNDFH II), lacto-N-neodifucohexaose II (LNnDFH II), para lacto-N-neohexaose (para-LNnH), lacto-N-neooctaose (LNnO), lacto-N-fucopentaose V (LNFP V), lacto-N-neofucopentaose V (LNnFP V), 3′-sialyl-3-fusoyl lactose (F-SL), LS-tetrasaccharide a (LSTa), 3′-sialyllactose (3′-SL), or combinations thereof.

The composition according to the present invention is an optimally suitable addition to a sports diet and allows full training without impacting intestinal health. Moreover, recovery is optimised, and the risk of infections is minimised.

Preferred embodiments of the composition are given in dependent claims 2 to 14.

A second aspect concerns a method for supporting a subject, and/or improving a subject's sports performance, and/or non-therapeutically improving a subject's recovery, according to claim 15. The method comprises determining whether a subject is a secretor or non-secretor of an HMO; and administering an effective dose of an HMO to the subject.

Preferred embodiments of the method are given in dependent claims 16 to 25.

A third aspect relates to a composition comprising at least one human milk oligosaccharide (HMO) according to claim 26. Preferred embodiments are shown in the dependent claims 27 to 29.

### DETAILED DESCRIPTION

In a first aspect, the present invention relates to a composition for use in supporting and/or improving intestinal health in athletes.

Unless otherwise defined, all terms used in the description of the invention, including technical and scientific terms, have the meaning as commonly understood by a person skilled in the art to which the invention pertains. For a better understanding of the description of the invention, the following terms are explained explicitly.

In this document, ‘a’ and ‘the’ refer to both the singular and the plural, unless the context presupposes otherwise. For example, ‘a segment’ means one or more segments.

When the term ‘around’ or ‘about’ is used in this document with a measurable quantity, a parameter, a duration or moment, and the like, then variations are meant of approx. 20% or less, preferably approx. 10% or less, more preferably approx. 5% or less, even more preferably approx. 1% or less, and even more preferably approx. 0.1% or less than and of the quoted value, insofar as such variations are applicable in the described invention. However, it must be understood that the value of a quantity used where the term ‘about’ or ‘around’ is used, is itself specifically disclosed.

The terms ‘comprise’, ‘comprising’, ‘consist of’, ‘consisting of’, ‘provided with’, ‘include’, ‘including’, ‘contain’, ‘containing’, are synonyms and are inclusive or open terms that indicate the presence of what follows, and which do not exclude or prevent the presence of other components, characteristics, elements, members, steps, as known from or disclosed in the prior art.

Quoting numerical intervals by endpoints comprises all integers, fractions and/or real numbers between the endpoints, these endpoints included.

A first aspect of the present invention relates to a composition for use in supporting and/or improving intestinal health in athletes, wherein the composition comprises an effective dose of at least one human milk oligosaccharide (HMO) selected from the group of 2′-fucosyllactose (2′-FL), 3-fucosyllactose (3-FL), lactodifucotetraose (DF-L), lacto-N-tetraose (LNT), lacto-N-neotetraose (LNnT), lacto-N-fucopentaose I (LNFP I), lacto-N-difucohexaose II (LNDFH II), lacto-N-neodifucohexaose II (LNnDFH II), para lacto-N-neohexaose (para-LNnH), lacto-N-neooctaose (LNnO), lacto-N-fucopentaose V (LNFP V), lacto-N-neofucopentaose V (LNnFP V), 3′-sialyl-3-fusoyl lactose (F-SL), LS-tetrasaccharide a (LSTa), 3′-sialyllactose (3′-SL), or combinations thereof.

In the context of the present invention, the term ‘human milk oligosaccharides’ (HMOs) refers to sugar molecules that occur naturally in human breast milk. HMOs have a prebiotic effect in the newborn child and are essential for the development of a healthy intestinal flora. HMOs in the context of the present invention are available from both a natural and synthetic source.

An ‘effective dose’ in the context of the present invention designates an ‘active dose’ and can be interpreted as the minimal dose at which the beneficial effects of the administered composition are manifested.

The term ‘intestinal health’ refers to the gastrointestinal microbiota, also microbiome, or the microorganisms that are present in the gastrointestinal tract. An individual's gastrointestinal microbiota can contain up to 30,000 different types of bacteria, some of which perform important or even necessary functions, while others are more harmful to the well-being of the subject in which they are present. Good intestinal health implies a healthy balance between the various microorganisms in the digestive system and promotes the general health of the subject.

The term ‘athlete’ can be interpreted as any individual who engages in sports activities. Sports activities according to the present invention comprise activities from the group of, but are not limited to, swimming, football, karate, hockey, strength training, weightlifting, shot-putting, gymnastics, athletics, cycling, running, sprinting, hurdling, skating, rowing, long jump, boxing, skiing, anaerobic training, aerobics, or combinations thereof.

The present invention, and more specifically the presence of an effective dose of at least one human milk oligosaccharide, has the advantage of effectively supporting intestinal function in a subject, preferably an athlete. The target group of the present invention is particularly sensitive to disorders of the gastrointestinal tract, as athletes regularly put their bodies to the limit. Since a large amount of catecholamines are released when delivering intensive sports performance, this can make a significant contribution to impaired intestinal health. For example, catecholamines can dramatically increase the growth of Gram-negative bacteria such as Escherichia coli and Yersinia enterocolitica. Enteric bacteria's ability to respond directly to stress-related neuromediators such as catecholamines acts at the level of the intestinal mucosa, which represents a complex network of epithelial cells, immunocytes and nerves in direct contact with the external environment. The composition according to the present invention supports and/or improves intestinal health thereby reducing the negative impact of catecholamines. The composition according to the present invention is an optimally suitable supplement to a sports diet and allows the athlete to exercise fully without impacting intestinal health. In addition, the athlete is better and faster able to recover after an intensive workout.

The term ‘pathogenicity’ denotes the potential pathogenic potential of an organism or substance. Typically, the term ‘pathogen’ is used to describe an infectious microorganism or agent, such as a virus, bacteria, protozoa, or fungus.

‘Catecholamines’ are neurotransmitters produced by the adrenal glands and the postganglionic fibres of the sympathetic nervous system. They comprise dopamine, epinephrine (adrenaline) and norepinephrine. Each of these neutrotransmitters is broken down into breakdown products which are excreted in the urine. Catecholamines are usually released into the bloodstream as a result of physical or emotional stress, where dopamine and norepinephrine in particular also exert a hormonal effect. They are produced in significant quantities when delivering intensive sports performance and have an indirect negative impact on intestinal health.

‘Epithelial tissue’ or ‘epithelium’ is the tissue that lines the outer surfaces of organs and blood vessels in the body, as well as inner surfaces of internal organs. Epithelial tissue forms a large surface that is in direct contact with the microbial flora present in the intestinal tract.

Preferably, it comprises human milk comprises oligosaccharide (HMO) 2′-fucosyllactose (2′-FL). The effective action of 2′-FL is based on its resemblance to certain pathogenic receptors found on the gut wall. Consequently, in various cases 2′-FL acts as an analogue of these pathogenic receptors, significantly reducing the risk of infection. HMOs, and in particular 2′-FL, have a direct effect on the epithelial structure of the intestines and interfere with the adhesion of various pathogenic bacteria, toxins, parasites and viruses. According to an embodiment, the HMOs interfere with bacteria from the group of Campylobacter jejuni, Escherichia coli, Vibrio cholerae, Salmonella fyris, Helicobacter pylori, or combinations thereof.

According to a further or other embodiment, the human milk oligosaccharide (HMO) comprises 2′-fucosyllactose (2′-FL) and/or derivatives thereof, which derivatives retain at least 70% of the biological functions of native 2′-FL. Preferably at least 80% of the biological functions are retained, more preferably at least 90%, most preferably at least 99%. Said biological functions of 2′-FL comprise its beneficial effects on the intestinal function, such as anti-inflammatory effects, antibacterial adhesion effects, prebiotic effects, etc.

The human milk oligosaccharides (HMOs) described herein can be prepared by any technique known in the art. According to an embodiment, the HMOs can be produced synthetically. Synthetic production of HMOs is possible, for instance, through microbial fermentation, enzymatic processes, chemical synthesis, or combinations thereof. According to some embodiments, the HMOs are synthesised by means of recombinant microorganisms such as Escherichia coli and/or Corynebacterium glutamicum.

According to a further or other embodiment, the effective dose of the human milk oligosaccharide (HMO) is comprised between 0.1 and 1.5 g/day or between 0.5 and 5.0 g/day. Within this range, the present composition acts as a curative and/or preventative agent for reduced intestinal motility and painful intestines. Athletes create an increased dose of norepinephrine in delivering long-lasting and intensive sports performance, which has the function of mobilising the brain and body for action. One of the effects of norepinephrine is to reduce blood flow to the gastrointestinal system and inhibit bladder and gastrointestinal motility. This inhibition of motility by norepinephrine can contribute to reduced intestinal health. The composition according to the present invention offers a solution for this, since it optimally stimulates and/or supports intestinal motility.

Preferably, the effective dose of the human milk oligosaccharide (HMO) is comprised between 0.3 and 1.0 g/day or between 1.8 and 2.2 g/day. The dose is in particular suitable when using sports supplements containing a high content of fructose and/or artificial sweeteners. The frequent use of fructose in sports drinks and supplements has already been linked to reduced intestinal health. Fructose reaches both the liver and the gastrointestinal microbiota, fueling pathogenic bacteria and potentially overgrowing beneficial bacteria. Consequently, the risk of infections is greater. The present composition decreases pathogenicity as the binding of pathogens to receptors on the epithelial cells in the intestinal tract is decreased. Pathogens or toxins bind to the HMOs and are eventually removed from the gastrointestinal tract. This significantly reduces the risk of (pathogenic) infections.

A specific advantage of the composition according to any of the previous embodiments is the inhibition of lipopolysaccharide (LPS) induced inflammation. Gram-negative, pathogenic bacteria are able to activate mucosal inflammation by binding the lipopolysaccharide matrix (LPS) to receptors present on the gut wall. The composition according to the present invention is capable of attenuating said LPS-induced inflammation through modulation of the expression of the CD14 gene. The CD14 gene encodes a protein that is critical for the proper functioning of the immune system and counteracts LPS-induced inflammation. Thus, the composition according to the present invention contributes to the athlete's general well-being, since the risk of bacterial infections is reduced, LPS-induced inflammation is inhibited, and intestinal motility is stimulated. The increased well-being of the athlete directly leads to significantly improved sports performance and a reduced recovery time.

Use of the composition in supporting and/or improving intestinal health in athletes, according to some embodiments, comprises increased production of short chain fatty acids (SCFAs) in the intestines. The composition, in particular the human milk oligosaccharide contained herein, has the advantage that it is a suitable substrate for Bifidobacterium bifidum, Bacteroides fragilis, Bacteroides vulgatus, or combinations thereof, among others. These microorganisms are responsible for the production of the said short chain fatty acids and are thus capable of lowering the pH in the intestines. This slows down the growth of unwanted bacteria as described further below. The increased production of short chain fatty acids has the additional advantage that it produces an increased gene expression in the gut wall. The induction of histone hyperacetylation by short chain fatty acids may play a beneficial role in this.

‘Lipopolysaccharides’, or ‘lipoglicans’, are polymeric molecules comprising a lipid moiety, a polysaccharide moiety and the so-called ‘O antigen’. They occur as the outer membrane of Gram-negative bacteria, which interact with receptors on the gut wall.

According to a further or alternative embodiment of the composition, the athletes have followed a course of antibiotics before administration of the composition. Taking a course of antibiotics is detrimental to the athlete's intestinal health. After the course of treatment, the athlete is therefore more susceptible to new infections, which significantly undermine the athlete's performance and recovery.

Before administration of the composition, it is determined according to an embodiment whether the athletes are secretor or non-secretor of said HMO. Non-secretors are not naturally capable of producing said HMO, and are therefore particularly sensitive to intestinal health problems and associated risks, as described in previous embodiments.

According to an embodiment, prior to administration of the composition, athletes are tested for the presence or absence of one or more genetic polymorphisms or mutations. Such polymorphisms or mutations in the genetic material can be indicators of reduced or absent production of HMOs and thus may be indicators of an increased risk of decreased intestinal health, pathogenic infections, decreased gut motility or even chronic intestinal disorders.

Preferably, said polymorphisms or mutations concern the FUT2 gene. The FUT2 gene is involved in the production of 2′-FL, where individuals with an inactivating polymorphism in the FUT2 gene are called ‘non-secretors’. Non-secretors are deficient in innate intestinal carbohydrates that contain fucose, which makes them extra susceptible to intestinal dysbiosis. The term ‘intestinal dysbiosis’ denotes an imbalance between different microorganisms present in the intestinal system. In addition, non-secretors have a higher chance of developing intestinal disorders such as Crohn's disease.

The polymorphisms mentioned are in particular the single nucleotide polymorphisms (SNPs) rs601338, rs492602, rs516246 and/or rs602662. SNPs according to this numbering can be found on databases such as SNPedia.

According to an embodiment, the composition further comprises one or more excipients, wherein the HMO and the excipients are in a ratio comprised between 1:8 and 6:8. In this light, the composition can also be interpreted as a food supplement, or sports supplement. A ratio of 1:8 to 6:8 allows the HMO, and in particular the 2′-FL, to act optimally towards one or more of its effects, the one or more excipients providing the composition with additional, beneficial effects. Moreover, within the present ratios, the composition forms little to no additional burden on the gastrointestinal system and is optimally absorbed by the subject. Preferably, the 2′-fucosyllactose and the one or more excipients are in a ratio comprised between 1:8 and 3:8. The present proportions allow the addition of more excipients to the composition, whereby several additional effects are obtained.

According to an embodiment, the one or more excipients are selected from the group of amino acids, monosaccharides, micronutrients, antioxidants, vitamins, vegetable extracts, fibres, derivatives of any of the previous excipients, or combinations thereof.

According to an embodiment, the vitamins are selected from the group of vitamin A, vitamin D, vitamin E, vitamin K, thiamine, riboflavin, pyridoxine, vitamin C, carotenoids, niacin, folic acid, pantothenic acid, biotin, choline, inositol, their salts and derivatives, and combinations thereof. The excipients according to the present invention contribute to the proper functioning of the gastrointestinal system, support intestinal health and support the general well-being of the subject, in particular the athlete.

According to an embodiment, the present invention comprises vegetable extracts selected from the group of ginger extract, soy extract, garlic extract, echinacea extract, ginseng extract, St. John's wort extract, elderberry extract, cranberry extract, turmeric extract, Ginkgo biloba extract, or combinations thereof. They are a natural source of polyphenols, and may act as an antioxidant and/or support the immune system and athletic performance. Preferably, the composition comprises cranberry extract. The use of cranberry extract in the present composition is particularly advantageous since cranberry extract acts as proanthocyanidin and is capable of triggering beneficial bacterial strains such as Akkermansia muciniphila.

According to some embodiments, the composition comprises one or more polyphenols, preferably proanthocyanidins, to stimulate the growth of Akkermansia muciniphila in the intestinal microbiome. This addition influences, in particular by the said proliferation of Akkermansia, various metabolic processes, with a favourable effect on the body composition. In particular, the proliferation of Akkermansia was associated with maintaining a low fat percentage. Considering the sports target group of the present invention, this is an important additional advantage of the composition.

In the context of the present invention, the amino acids preferably comprise glutamine, and branched-chain amino acids (BCAAs), namely leucine, isoleucine and/or valine. During intensive training they can be used directly as an energy source. In addition, they contribute, especially leucine, to activation of muscle protein synthesis. In combination with the HMOs of the present invention, the recovery time is further shortened.

‘Antioxidants’ are components capable of capturing free radicals and therefore lowering oxidative stress. According to an embodiment, the composition comprises one or more antioxidants suitable for oral administration, including vitamin A, vitamin E, vitamin C, retinol, tocopherol, carotenoids, including lutein, beta-carotene, zeaxanthin, lycopene, and combinations thereof.

According to an embodiment, said micronutrients comprise calcium, phosphorus, sodium, chloride, magnesium, manganese, iron, copper, zinc, selenium, iodine, chromium, molybdenum, or combinations thereof.

The composition, or also the food supplement or the sports supplement, according to the present invention further comprises according to an embodiment one or more proteins, carbohydrates and/or fats. The food supplement can be used as a supplement to a healthy diet, and can support the athlete in carrying out intensive efforts and/or sports performance. According to an embodiment, the composition comprises one or more protein sources, which have a total concentration in the supplement comprised between 40.0 and 80.0 m %, preferably between 50.0 and 80.0 m %, more preferably between 60.0 and 80.0 m %, even more preferably between 65.0 and 80.0 m %.

The composition according to the present invention can be formulated in a form adapted to the chosen route of administration, including, but not limited to, oral or parental, including subcutaneous, intramuscular, intraperitoneal, intratumoral and intravenous administration. ‘Oral administration’, as defined herein, comprises any form of administration in which the composition is absorbed via the subject's oesophagus. Oral formulations comprise any solid, liquid or powder formulation suitable for use herein, with the proviso that such formulation allows for the safe and effective oral delivery of the effective dose of 2′-fucosyllactose (2′-FL).

According to an embodiment, the composition is a soluble powder. The soluble powder has the advantage that the composition can be stored in a suitable container of only limited size and can be dissolved in a liquid by the consumer. The composition not only takes up a small volume, but it also has a long shelf life.

According to an embodiment, the composition is directly ingestible as a capsule. A capsule according to the present embodiment preferably comprises between 0.1 and 1.5 g of 2′-fucosyllactose (2′-FL). Intake of the effective dose is accomplished through daily intake of 1 to 10 capsules. More preferably, a capsule comprises between 0.3 and 1.0 g of 2′-fucosyllactose (2′-FL), even more preferably between 0.3 and 0.8 g of 2′-FL, between 0.3 and 0.7 g of 2′-FL, between 0.3 and 0.6 g of 2′-FL, most preferably between 0.3 and 0.5 g of 2′-fucosyllactose 2′-FL. A daily intake of 2 to 10 capsules, preferably from 2 to 9 capsules, more preferably from 2 to 8 capsules, even more preferably from 2 to 7 capsules, most preferably from 2 to 6 capsules, provides for intake of the effective dose. In another embodiment, the composition is ingestible through food products and/or drinks.

According to a preferred embodiment, the composition is a sports drink. A sports drink brings a great ease of use for the consumer, since no actions are required immediately before ingestion by the consumer.

In this light, the present invention also relates to a method of supporting and/or improving intestinal health in athletes, wherein a composition is administered comprising an effective dose of at least one human milk oligosaccharide (HMO) selected from the group of 2′-fucosyllactose (2′-FL), 3-fucosyllactose (3-FL), lactodifucotetraose (DF-L), lacto-N-tetraose (LNT), lacto-N-neotetraose (LNnT), lacto-N-fucopentaose I (LNFP I), lacto-N-difucohexaose II (LNDFH II), lacto-N-neodifucohexaose II (LNnDFH II), para lacto-N-neohexaose (para-LNnH), lacto-N-neooctaose (LNnO), lacto-N-fucopentaose V (LNFP V), lacto-N-neofucopentaose V (LNnFP V), 3′-sialyl-3-fusoyl lactose (F-SL), LS-tetrasaccharide a (LSTa), 3′-sialyllactose (3′-SL), or combinations thereof. According to an embodiment, the effective dose is comprised between 0.5 and 5.0 g/day. In particular, the method comprises administering a composition according to any of the preceding embodiments.

In a second aspect, the invention relates to a method for supporting a subject, and/or improving a subject's sports performance, and/or non-therapeutically improving a subject's recovery, wherein it is determined whether the subject is a secretor or non-secretor of an HMO, preferably 2′-FL, and wherein the subject is administered a composition comprising an effective dose of at least one human milk oligosaccharide (HMO) selected from the group of 2′-fucosyllactose (2′-FL), 3-fucosyllactose (3-FL), lactodifucotetraose (DF-L), lacto-N-tetraose (LNT), lacto-N-neotetraose (LNnT), lacto-N-fucopentaose I (LNFP I), lacto-N-difucohexaose II (LNDFH II), lacto-N-neodifucohexaose II (LNnDFH II), para lacto-N-neohexaose (para-LNnH), lacto-N-neooctaose (LNnO), lacto-N-fucopentaose V (LNFP V), lacto-N-neofucopentaose V (LNnFP V), 3′-sialyl-3-fusoyl lactose (F-SL), LS-tetrasaccharide a (LSTa), 3′-sialyllactose (3′-SL), or combinations thereof.

Preferably, the HMO comprises 2′-fucosyllactose (2′-FL).

According to an embodiment, it is determined whether the subject is a secretor or non-secretor of an HMO by monitoring for the presence or absence of one or more genetic polymorphisms or mutations in the 2-alpha-L-fucosyl transferase 2 (FUT2) gene.

In another embodiment, the subject's secretor/non-secretor status is determined by checking for the presence or absence of one or more genetic polymorphisms or mutations in a gene other than the 2-alpha-L-fucosyl transferase 2 (FUT2) gene.

According to a further or alternative embodiment, the secretor/non-secretor status of the subject is determined by checking for the presence or absence of one or more polymorphisms at the protein level, in particular the protein encoded by the FUT2 gene. The protein encoded by FUT2 is a Golgi membrane stacking protein involved in the production of a precursor of the H antigen required for the final step in the soluble A and B antigen synthesis pathway. This gene is one of two genes encoding the 2-alpha-L-fucosyltransferase 2 enzyme. SNPs at the level of the FUT2 gene and/or polymorphisms in the protein encoded by this gene lead to a reduced to absent secretion of 2′-FL and are therefore extremely suitable for determining the secretor/non-secretor status with regard to 2′-FL.

More preferably, the method comprises a DNA analysis, wherein single nucleotide polymorphisms (SNPs) in the 2-alpha-L-fucosyltransferase 2 (FUT2) gene are identified. The FUT2 gene is involved in the production of 2′-FL, where individuals with an inactivating polymorphism in the FUT2 gene are called ‘non-secretors’. Non-secretors are deficient in innate intestinal carbohydrates that contain fucose, which makes them extra susceptible to intestinal dysbiosis. The term ‘intestinal dysbiosis’ denotes an imbalance between different microorganisms present in the intestinal system. In addition, non-secretors have a higher chance of developing intestinal disorders such as Crohn's disease.

The single nucleotide polymorphisms (SNPs) preferably comprise the SNPs rs601338, rs492602, rs516246 and/or rs602662. Such SNP numbering can be consulted on databases such as SNPedia. The SNPs of the present embodiment are exceptionally relevant for the non-secretor status of a subject with respect to 2′-FL. Preferably SNP rs601338 is detected, of which 25 to 30% of the population is a carrier. More preferably, at least 2 SNPs from the group of rs601338, rs492602 and/or rs602662 are detected. Even more preferably, rs601338, rs492602 and rs602662 are detected. DNA analysis and/or the detection of SNPs according to the present invention is performed by standard techniques as known from the prior art. Preferably, a microarray or a single nucleotide polymorphism (SNP) chip is used to identify SNPs present in the FUT2 gene.

According to an embodiment of the method, the effective dose of the HMO is optionally determined based on the secretor/non-secretor status, comprised between 0.5 and 5.0 g/day. The action of HMOs, and in particular 2′-FL, is multifold and comprises interference in pathogen and toxin binding, thereby reducing the risk of infection; and improves gut motility. The various effects are clearly expressed within the range mentioned.

According to an embodiment, the effective dose of the HMO, especially 2′-fucosyllactose (2′-FL), in a non-secretor is at least 1.8 g/day. Preferably, the effective dose is comprised between 1.8 and 4.0 g/day, which dose is optimally suited for improving intestinal health in non-secretors of HMOs. The risk of intestinal disorders is generally considerably higher in non-secretors than in secretors. Administration of the effective dose of HMO reduces the risk of intestinal disorders, through inhibition of pathogens and improvement of gut motility. The effective dose is preferably comprised between 2.0 and 3.5 g/day, more preferably between 2.0 and 3.0 g/day.

An embodiment comprises an effective dose of the HMO, especially 2′-fucosyllactose (2′-FL), in a secretor at least 0.8 g/day. Preferably, the effective dose is comprised between 0.8 and 3.0 g/day, more preferably comprised between 1.0 and 2.5 g/day, even more preferably comprised between 1.8 and 2.2 g/day.

The various embodiments as described herein also relate to the use of a composition according to the present invention for the manufacture of a drug for supporting and/or improving intestinal health in a subject. Preferably, the subject is an athlete.

Use of the composition in supporting a subject, and/or improving a subject's sports performance, and/or non-therapeutically improving a subject's recovery comprises, according to some embodiments, an increased production of short chain fatty acids (SCFAs) in the intestines. The increased production of short chain fatty acids has the advantage that it produces an increased gene expression in the intestinal wall. The induction of histone hyperacetylation by short chain fatty acids may play a beneficial role in this. Since gene expression in the intestinal wall is closely linked to mitochondrial biogenesis, an increased production of mitochondria in the athlete is also realised by using the present composition. Increased production of mitochondria, which play an essential role in energy production and regulation, is of great added value in athletes, especially endurance athletes. Such a composition is extremely beneficial for supporting and improving sports performance, as well as for improving recovery in an athlete.

According to some embodiments, the use of the composition in supporting a subject, and/or improving a subject's sports performance, and/or non-therapeutically improving a subject's recovery, comprises improving synaptic plasticity. This is particularly advantageous for athletes who practice sports of a certain technical level of difficulty and supports them in learning and/or improving complex sports skills.

According to an embodiment of the method, the effective dose of HMO is administered via a composition according to an embodiment of the first aspect of the invention.

According to an embodiment, the subject is an athlete. Since performing intensive workouts has a major impact on the microbiota, including the microbiome, athletes in particular benefit from a composition and method according to the present invention.

Preferably, the athlete has followed a course of antibiotics prior to the method. Following a course of antibiotics has an adverse effect on intestinal health, which makes the method according to the present invention particularly advantageous.

A third aspect of the present invention relates to a composition comprising at least one human milk oligosaccharide (HMO) selected from the group of 2′-fucosyllactose (2′-FL), 3-fucosyllactose (3-FL), lactodifucotetraose (DF-L), lacto-N-tetraose (LNT), lacto-N-neotetraose (LNnT), lacto-N-fucopentaose I (LN FP I), lacto-N-difucohexaose II (LNDFH II), lacto-N-neodifucohexaose II (LNnDFH II), para lacto-N-neohexaose (para-LNnH), lacto-N-neooctaose (LNnO), lacto-N-fucopentaose V (LNFP V), lacto-N-neofucopentaose V (LNnFP V), 3′-sialyl-3-fusoyl lactose (F-SL), LS-tetrasaccharide a (LSTa), 3′-sialyllactose (3′-SL), or combinations thereof. The composition herein exhibits all previously discussed advantages with respect to a subject.

Preferably, the composition is formulated as a powder, more preferably a water-soluble powder. A powder according to the present composition is easy to dose based on the needs of the subject. According to some embodiments, the composition comprises between 1.0 and 50.0 g of human milk oligosaccharide per 100 g of composition. Preferably, the human milk oligosaccharide (HMO) is 2′-fucosyllactose (2′-FL). In what follows, the invention is described by way of non-limiting examples illustrating the invention, and which are not intended to and should not be interpreted as limiting the scope of the invention.

### EXAMPLES

The invention will now be further elucidated by means of the following examples, without, however, being limited thereto.

### Example 1: Composition for Improving Intestinal Health

The following composition is ideal for improving and/or supporting intestinal health, with a recommended daily dose of 3.0 g/day.

| ingredient | concentration (m %) |
|---|---|
| 2′-fucosyllactose (2′-FL) | 70.00 |
| lacto-N-tetraose (LNT) | 15.00 |
| lacto-N-fucopentaose V (LNFP V) | 10.00 |
| various fillers | 5.00 |

Subjects who receive the composition on a daily basis are less susceptible to infections, feel fitter and experience improved intestinal motility.

### Example 2: Composition for Optimising Recovery in Endurance Athletes

The composition shown in the table below optimises recovery in athletes by supporting intestinal health from a daily dose of 2.5 g/day.

| ingredient | concentration (m %) |
|---|---|
| 2′-fucosyllactose (2′-FL) | 80.00 |
| various fillers | 20.00 |

In 20 endurance athletes who were administered the composition at 2.5 g/day for 60 days, the required recovery time after intensive training was shortened by 5 to 10% compared to the control group (20 subjects). Athletes who received the composition experienced less abdominal and intestinal complaints during and/or after intensive training and indicated that they felt recovered more quickly.

Twenty-five percent of the athletes tested indicated that they were also better able to schedule intensive training sessions that followed each other in quick succession.

### Example 3: Powder-Formed Food Supplement for Athletes

The food supplement with composition as shown in the following table is a suitable addition to a healthy diet, improving intestinal health, improving recovery and sports performance.

| ingredient | concentration (m %) |
|---|---|
| 2′-fucosyllactose (2′-FL) | 25.00 |
| micronutrients (Zn salt) | 0.10 |
| L-glutamine | 6.30 |
| vegetable extracts | 65.00 |
| various fillers | 3.60 |

The food supplement is powdery and should be dissolved in water, milk, orange juice or another drinkable liquid. For daily consumption, preferably a dose of 8 g is dissolved in a liquid volume of 100 mL.

Daily consumption improves the immune system in athletes, as a result of improved intestinal health. In particular, when other supplements are used, especially supplements comprising fructose and/or artificial sweeteners, optimal gut motility is obtained, and the risk of pathogenic infections is minimised.

### Example 4: Protein Powder with Human Milk Oligosaccharides

The protein powder with the composition below supports an optimal build-up of muscle mass and optimises the required recovery time between workouts, allowing a higher training volume.

| ingredient | concentration (m %) |
|---|---|
| whey protein | 68.00 |
| carbohydrates | 16.00 |
| 2′-fucosyllactose (2′-FL) | 4.50 |
| calcium (Ca) | 0.30 |
| potassium (K) | 0.40 |
| various additives | 10.80 |

With daily consumption of 45 g, preferably immediately after a strength training, the build-up of muscle mass is maximised and the negative side-effects of an intensive training session on the gastrointestinal tract minimised. The protein powder is suitable for a solution of 200 to 300 mL of drinkable liquid. The present powder shortens the required recovery time after a workout, which is particularly advantageous if a training regimen of 4 to 6 sessions per week is envisaged.

### Example 5: Improving Intestinal Health in Non-Secretors of HMOs

Regarding non-secretors of human milk oligosaccharides, the invention provides a method for improving intestinal health in these subjects, which goes through the following steps.
1. Determination of the 2′-fucosyllactose secretor/non-secretor status of the subject by detecting the single nucleotide polymorphisms (SNPs) rs601338, rs492602, rs516246 and/or rs602662 at the 2-alpha-L-fucosyl transferase 2 (FUT2) gene and comprising the steps:
   a. taking a sample from the subject (blood, saliva, urine, hair, skin, etc.;
   b. isolation and purification of genomic DNA from the blood sample;
   c. isolation and purification of a DNA fragment comprising the 2-alpha-L-fucosyl transferase 2 (FUT2) gene by restriction enzymes;
   d. optional replication of the relevant DNA fragment by PCR;
   e. detection of the SNPs by microarray genotyping, i.e. a single nucleotide polymorphism (SNP) chip according to rs601338, rs492602, rs602662 and/or possibly other relevant SNPs;
   f. if one or more SNPs give a positive result, the subject is characterised as a non-secretor, otherwise the subject is characterised as a 2′-FL secretor.
2. Optionally, determination of the effective dose of 2′-fucosyllactose (2′-FL) based on secretor/non-secretor status:
   a. for non-secretors, a daily dose comprised between 2.0 and 3.0 g is prescribed in order to minimise the specific risks to which this group is exposed;
   b. for secretors, a daily dose comprised between 1.8 and 2.2 g/day is prescribed to support normal intestinal health.
3. Administration of the effective dose of 2′-fucosyllactose (2′-FL) to the subject. Depending on the determined secretor/non-secretor status, the subject is administered a dose between 2.25 and 3.75 g/day of the composition below.

| ingredient | concentration (m %) |
|---|---|
| 2′-fucosyllactose (2′-FL) | 80.00 |
| various fillers | 20.00 |

This methodology provides for efficient support and/or improvement of intestinal health in non-secretors of 2′-FL, who are more likely to develop intestinal disorders such as Crohn's disease. The gastrointestinal microbiota is maximally supported and intestinal motility is stimulated. This reduces the chance of developing infections, and chronic diseases in the long term, in this target group.

1. Composition for use in supporting and/or improving intestinal health in athletes, characterised in that the composition comprises an effective dose of at least one human milk oligosaccharide (HMO) selected from the group of 2′-fucosyllactose (2′-FL), 3-fucosyllactose (3-FL), lactodifucotetraose (DF-L), lacto-N-tetraose (LNT), lacto-N-neotetraose (LNnT), lacto-N-fucopentaose I (LNFP I), lacto-N-difucohexaose II (LNDFH II), lacto-N-neodifucohexaose II (LNnDFH II), para lacto-N-neohexaose (para-LNnH), lacto-N-neooctaose (LNnO), lacto-N-fucopentaose V (LNFP V), lacto-N-neofucopentaose V (LNnFP V), 3′-sialyl-3-fusoyl lactose (F-SL), LS-tetrasaccharide a (LSTa), 3′-sialyllactose (3′-SL), or combinations thereof.

2. Composition for use according to claim 1, characterised in that the human milk oligosaccharide (HMO) comprises 2′-fucosyllactose (2′-FL).

3. Composition for use according to claim 1, characterised in that the human milk oligosaccharide (HMO) is a synthetically produced HMO.

4. Composition for use according to claim 1, characterised in that the effective dose of the human milk oligosaccharide (HMO) is comprised between 0.1 and 1.5 g/day or between 0.5 and 5.0 g/day.

5. Composition for use according to claim 4, characterised in that the effective dose of the human milk oligosaccharide (HMO) is comprised between 0.3 and 1.0 g/day or between 1.8 and 2.2 g/day.

6. Composition for use according to claim 1, characterised in that supporting and/or improving intestinal health comprises an increased production of short chain fatty acids (SCFA) in the intestines.

7. Composition for use according to claim 1, characterised in that the athletes have followed a course of antibiotics prior to administration of the composition.

8. Composition for use according to claim 1, characterised in that prior to administration of the composition it was determined whether the athletes are secretor or non-secretor of said HMO.

9. Composition for use according to claim 1, characterised in that the athletes are tested for the presence or absence of one or more genetic polymorphisms or mutations prior to administration of the composition.

10. Composition for use according to claim 9, characterised in that said polymorphisms or mutations concern the FUT2 gene.

11. Composition for use according to claim 1, characterised in that the composition comprises one or more excipients, wherein the HMO and the one or more excipients relate according to a ratio which is comprised between 1:8 and 6:8.

12. Composition for use according to claim 11, characterised in that the one or more excipients are selected from the group of amino acids, monosaccharides, micronutrients, vegetable extracts, fibres, derivatives of any of the preceding excipients, or combinations thereof.

13. Composition for use according to claim 1, characterised in that the food supplement is a soluble powder.

14. Composition for use according to claim 1, characterised in that the food supplement is a sports drink.

15. Method for supporting a subject, improving a subject's sports performance, and/or non-therapeutically improving a subject's recovery, wherein it is determined whether the subject is a secretor or non-secretor of an HMO, and wherein the subject is administered a composition comprising an effective dose of at least one human milk oligosaccharide (HMO) selected from the group of 2′-fucosyllactose (2′-FL), 3-fucosyllactose (3-FL), lactodifucotetraose (DF-L), lacto-N-tetraose (LNT), lacto-N-neotetraose (LNnT), lacto-N-fucopentaose I (LNFP I), lacto-N-difucohexaose II (LNDFH II), lacto-N-neodifucohexaose II (LNnDFH II), para lacto-N-neohexaose (para-LNnH), lacto-N-neooctaose (LNnO), lacto-N-fucopentaose V (LNFP V), lacto-N-neofucopentaose V (LNnFP V), 3′-sialyl-3-fusoyl lactose (F-SL), LS-tetrasaccharide a (LSTa), 3′-sialyllactose (3′-SL), or combinations thereof.

16. Method according to claim 15, characterised in that the HMO comprises 2′-fucosyllactose (2′-FL).

17. Method according to claim 15, characterised in that it is determined whether the subject is a secretor or non-secretor of an HMO by checking for the presence or absence of one or more genetic polymorphisms or mutations in the 2-alpha-L fucosyl transferase 2 (FUT2) gene.

18. Method according to claim 17, characterised in that the method comprises a DNA analysis, wherein single nucleotide polymorphisms (SNPs) in the 2-alpha-L-fucosyl transferase 2 (FUT2) gene are identified.

19. Method according to claim 18, characterised in that the single nucleotide polymorphisms (SNPs) are rs601338, rs492602, rs516246 and/or rs602662.

20. Method according to claim 15, characterised in that the effective dose of the HMO in a non-secretor is comprised between 1.8 and 4.0 g/day.

21. Method according to claim 15, characterised in that the effective dose of the HMO in a secretor is comprised between 0.8 and 3.0 g/day.

22. Method according to claim 15, characterised in that supporting a subject, improving the sports performance of a subject, and/or non-therapeutically improving the recovery of a subject, increases production of short chain fatty acids (SCFAs) in the intestines.

23. Method according to claim 15, characterised in that supporting a subject, improving the sports performance of a subject, and/or non-therapeutically improving the recovery of a subject comprises improving synaptic plasticity.

24. Method according to claim 15, characterised in that the subject is an athlete.

25. Method according to claim 24, characterised in that the athlete has followed a course of antibiotics in advance.

26. Composition comprising at least one human milk oligosaccharide (HMO) selected from the group of 2′-fucosyllactose (2′-FL), 3-fucosyllactose (3-FL), lactodifucotetraose (DF-L), lacto-N-tetraose (LNT), lacto-N-neotetraose (LNnT), lacto-N-fucopentaose I (LNFP I), lacto-N-difucohexaose II (LNDFH II), lacto-N-neodifucohexaose II (LNnDFH II), para lacto-N-neohexaose (para-LNnH), lacto-N-neooctaose (LNnO), lacto-N-fucopentaose V (LNFP V), lacto-N-neofucopentaose V (LNnFP V), 3′-sialyl-3-fusoyl lactose (F-SL), LS-tetrasaccharide a (LSTa), 3′-sialyllactose (3′-SL), or combinations thereof.

27. Composition according to claim 26, characterised in that the composition is formulated as a powder.

28. Composition according to claim 26, characterised in that the composition comprises between 1.0 and 50.0 g human milk oligosaccharide per 100 g composition.

29. Composition according to claim 26, characterised in that the human milk oligosaccharide (HMO) comprises 2′-fucosyllactose (2′-FL).
